(19) United States
(12) Patent Application Publication
(10) Pub. No.: US 2025/0299325 A1
(43) Pub. Date: Sep. 25, 2025

(54) METHOD AND USE OF TRANSSCLERAL OPTICAL IMAGING FOR DETECTING A DISEASE

(71) Applicant: EarlySight SA, Geneva (CH)

(72) Inventors: Timothé Laforest, Geneva (CH); Mathieu Kunzi, Geneva (CH)

(73) Assignee: EarlySight SA, Geneva (CH)

(21) Appl. No.: 18/861,293

(22) PCT Filed: Apr. 30, 2023
(86) PCT No.: PCT/EP2023/061422

§ 371 Date: Oct. 29, 2024

(30) Foreign Application Priority Data
Apr. 30, 2022 (EP) 22171059.3

(51) Int. Cl. G06T 7/00; A61B 3/10; A61B 3/12; A61B 3/14

(52) U.S. Cl. CPC G06T 7/0012; A61B 3/102; A61B 3/12; A61B 3/14; G06T 2207/10101; G06T 2207/30041

(57) ABSTRACT

The present invention relates to a method of diagnosing, and/or prognosing a disease associated with an altered structure in the posterior segment of the eye, wherein the method comprises analyzing an image of the posterior segment of the eye obtained by a transscleral optical imaging (TOI) device for an altered structure relative to a reference, wherein the altered structure is indicative of the presence and/or progression the disease in a subject.

Diseases associated with alterations in the structure of the posterior segment of the eye, such as glaucoma, age-related macular degeneration (AMD) and diabetic retinopathy are the major cause of visual impairment worldwide. For example, an estimated 196 million people will be affected by age-related macular degeneration (AMD) in 2020. The posterior segment of the eye comprises the back two-thirds of the eye, including the vitreous humor, the retina, the choroid and the optic nerve. Of these, alterations in the retina, in particular the retinal neurons and the retinal pigment epithelium (RPE) are commonly associated with many diseases of the posterior segment of the eye.

The retina is the vitreal-most ten-layered light-sensitive nervous tissue membrane of the eye. Its role is to convert the received light stimuli into nerve impulses and send them with the optic nerve to the visual centres of the brain. The retinal pigmented epithelium (RPE) is the scleral-most monolayer of pigmented retinal cells.

Although they are located outside of the neurosensory retina, RPE cells play some crucial roles, such as light absorption, epithelial transport and maintenance of the visual cycle. Some RPE cell morphology characteristics, namely cell density, number of neighbors, eccentricity, and form factor, are postulated to differ depending on cell maturation and condition. Some other studies report RPE cell loss caused by diseases of the eye and aging.

Several diagnostic imaging modalities allow for in vivo assessment of the human eye (e.g. optical coherence tomography (OCT), scanning laser ophthalmoscopy (SLO), and fundus autofluorescence) these methods do not allow for the diagnosis of retinal diseases at their early stage because the minuscule changes in RPE cell morphology cannot be detected. Furthermore, RPE layer in vivo imaging at the single-cell level is challenging due to several factors, namely, the low contrast between neighboring cells, motion artefacts, retinal layer non-linearity, and difficulties with the image's focal point identification. The instruments used in eye clinics for routine eye fundus examination are not able to observe the minute changes in cell morphology that are present during early stages of the disease degenerative process.

Transscleral optical imaging (TOI), disclosed in 2017, is a novel non-invasive, in vivo, high-resolution imaging modality for posterior structures of the eye, in particular, the retina. The use of both adaptive optics and oblique illumination enhances the contrast of macroscopic and microscopic posterior segment structures, such as tissue structure, vasculature and RPE cells, The resultant superior imaging resolution enables very high resolution, including to the cellular level, e.g. discerning single RPE cells' cellular membranes.

The applicant described in WO/2017/195163 A1 disclosed a method for imaging a tissue of an eye, the method including the steps of providing oblique illumination to the eye by a plurality of light emitting areas of a light delivery device, the plurality of light emitting areas being independently controllable and arranged to direct light towards at least one of a retina and an iris of the eye, causing an output beam from light backscattered from the at least one of the retina and the iris by the oblique illumination, capturing the output beam with an imaging system to provide a sequence of images of a fundus of the eye, and retrieving a phase and absorption contrast image from the sequence of images of the fundus, wherein the sequence of images of the fundus of the step of capturing is obtained by sequentially turning on one or more of the plurality of light emitting areas at a time in the step of providing the oblique illumination. In other words, the method for oblique illumination, including transscleral illumination and transpalpebral illumination, allows for dark field and phase gradient techniques by using the scattering properties of the fundus. The oblique illumination, e.g transscleral oblique flood illumination, increases the contrast of many biological structures composing the retina layers and, coupled with adaptive optics high-resolution imaging, enables the observation of cells which play a key role in the diseases-related degenerative process. Obtaining a cellular-level high-resolution image enables a new view of the structure of the retina resulting in a better understanding of the degenerative retinal disease processes.

Further developments and elements of the TOI device by the applicants are disclosed in WO2020/121243 A1, WO2021/058367 A1 and WO2021/191331 A1. In WO2020/121243 A1 a TOI system with transscleral/transpalpebral illumination of the eye fundus was disclosed. The TOI system comprised a plurality of emitting areas; each of the emitting areas being configured to be independently controllable and directed towards the sclera of the intended eye to measure, providing transscleral oblique illumination of the eye fundus; an active eye aberration correcting system; and an imaging system configured to create multiple images of the eye fundus on multiple imaging sensors. In WO2021/191331 The light-delivering device was combined with optical coherence tomography (OCT) imaging.

The in vivo observation of the human retina at the cellular level is crucial to detect structural alterations before irreversible visual loss occurs, to follow the time course of retinal diseases and to evaluate and monitor the early effects of treatments. Despite the phenomenal advances in optical coherence tomography (OCT) and adaptive optics systems, in vivo imaging of several retinal cells is still elusive.

Laforest T. et al. “Transscleral Optical Phase Imaging of the Human Retina-TOPI” https://arxiv.org/abs/1905.06877 disclosed a transscleral optical imaging (TOI) device, which allows to image retinal cells with high contrast, high resolution, and within an acquisition time suitable for clinical use. TOI relies on high-angle oblique illumination of the retina, combined with adaptive optics, to enhance the phase contrast of transparent cells.

Given the lack in the art for methods which provide accurate detection of the early signs of structural alterations in the posterior eye segment which are associated with disease there is an urgent and unmet need in the art for methods capable of generating high resolution images of the posterior eye segment to allow the analysis of disease states is needed.

Accordingly, the present invention provides a new method for cellular resolution imaging of the posterior eye segment for the early diagnosis, prognosis and therapeutic susceptibility of diseases associated with alterations of the structure of the posterior eye segment.

Thus, the technical problem underlying the present invention is to provide a method for the early detection of structural alterations in the posterior segment of the eye which enables early diagnosis, prognosis and treatment of diseases associated with structural alterations of the posterior segment of the eye.

The invention, accordingly, relates to the following:
1. A method of diagnosing, and/or prognosing a disease associated with an altered structure in the posterior segment of the eye, wherein the method comprises analyzing an image of the posterior segment of the eye obtained by transscleral optical imaging (TOI) for an altered structure, wherein the altered structure is indicative of the presence and/or progression of the disease in a subject.
2. A method of treating a disease associated with an altered structure in the posterior eye segment, wherein the method comprises the steps of:
   a) analyzing an image of the posterior eye segment obtained by TOI for an altered structure, where the presence of an altered structure is indicative of the presence of the disease or the development of the disease in a subject; and
   b) administering to the subject identified as having or developing a disease according to step (a), an appropriate treatment for said disease.

3. The method according to item 1 or 2, wherein said altered structure is determined relative to a reference that is a TOI image of said posterior segment obtained from a similarly situated subject known not to have the disease or known not to be at risk of developing the disease.
4. A method for
   (i) evaluating the therapeutic effect in a subject of a treatment for a disease associated with an altered structure in the poster segment of the eye, or
   (ii) determining a subject's compliance with a prescribed treatment for a disease associated with an altered structure in the posterior segment of the eye,
      said method comprising analyzing a first and second image of the posterior segment of the eye of said subject obtained by TOI,
(a) wherein said first image is to be obtained before said treatment or prior to second image;
(b) wherein said second image is to be obtained after treatment or subsequent to said first image;
   (c) wherein the analyzing the first and second image is an analysis and comparison of the structure of the posterior segment in (a) and (b);
      wherein the maintenance of or decrease in an altered structure between (a) and (b) is indicative of said treatment having therapeutic effect or said subject complying with said treatment.
5. The method according to item 4, wherein said altered structure is a structure altered relative to that determined from analysis of a TOI image of said posterior segment obtained from a similarly situated subject known not to have the disease or known not to be at risk of developing the disease.
6. The method of item 4 wherein determination of the maintenance of the altered structure between (a) and (b) is
(d) a determination where said altered structure is substantially unchanged between (a) and (b) of item 4; or
(e) a determination where any further alteration or progression of said altered structure between (a) and (b) of claim 4 is not as severe or advanced as that between a set of reference first and second TOI images of said posterior segment obtained from a similarly situated subject known to have the disease and which subject has not been treated for said disease, wherein the reference first and second images were obtained or are to be obtained at the same interval as the first and second images of (a) and (b) of item 4.
7. The method according to any one of items 4 to 6, wherein said second image is to be obtained at least 2 days and no more than 730 days subsequent to said first image.
8. The method according to any one of items 1 to 7, wherein said disease associated with an altered structure in the posterior segment of the eye is uveitis, glaucoma, macular edema, macular hole, macular pucker, diabetic macular edema, diabetic retinopathy, diabetic eye diseases, retinopathy, age-related macular degeneration (AMD), wet AMD, dry AMD, early AMD, intermediate AMD, central serous chorioretinopathy, scleritis, optic nerve degeneration, geographic atrophy, choroidal disease, ocular sarcoidosis, optic neuritis, choroidal neovascularization, retinitis pigmentosa, retinal tears, Stargardt disease, ocular cancer, retinitis, corneal ulcers, cataract, infection with a virus (such as cytomegalovirus, herpes simplex, herpes zoster), infection with fungi (such as histoplasmosis), parasitic infection (such as toxoplasmosis, toxocariasis), bacterial infection (such as tuberculosis, syphilis), sarcoidosis, retinal vein occlusion, central retinal vein occlusion, branch retinal vein occlusion, retinal vascular disease, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, iridocyclitis diabetic retinopathy, retinopathy of prematurity (ROP), ischemic vasculopathies, inherited retinal dystrophies, retinal detachment, aberrant angiogenesis, retinal angiomatous proliferation (RAP), intraretinal microvascular abnormalities, pre-retinal neovascularization, choroidal angiogenesis, choroidal vasculopathy stroke, hypertension, diabetes, cardiovascular disease, prematurity, and papilloedema.

9. The method according to any one of items 1 to 8, wherein the image of said posterior segment of the eye is an image of the choroid, the choriocapillaris, Bruch's membrane, retinochoroidal tissue, the neuroretinal tissue, the nerve fiber layer,, the retinal pigment epithelium (RPE), the photorepectors, the ganglion cell layer, the retinal vasculature, the subretinal space, the retina, the macula, the lamina cribrosa, the optic disc or the optic nerve.
10. The method according to any one of items 1 to 9, wherein said altered structure is an alteration in the tissue structure.
11. The method according to item 10, wherein the alteration in tissue structure is an alteration in cell pattern, cell density, cell size, cell distribution or cell reflectivity.
12. The method according to item 10, wherein said alteration in tissue structure is an alteration in tissue reflectivity that is an alteration in hyporeflective regions, hyperreflective regions, hyporeflective regions within a hyperreflective region, or any combination thereof.
13. The method according to any one of items 1 to 12, wherein said image is an image of the RPE.
14. The method according to any one of items 1 to 12, wherein said image is an image of the choriocapillaris.
15. The method according to any one of items 1 to 12, wherein the image is an image of the nerve fiber layer, optic disc and/or retinal vasculature.
16. The method according to item 13, wherein said disease is AMD and said altered structure is an alteration in RPE cell density, RPE cell size, hyperreflective regions, hyporeflective regions, and/or hyporeflective regions within a hyperreflective region.
17. The method according to item 13 or 14, wherein said disease is central serous chorioretinopathy and said altered structure is an alteration of the RPE and the choriocapillaris.
18. The method according to item 15, wherein said disease is glaucoma and said altered structure is an alteration of the nerve fiber layer (including but not limited to alteration of the orientation of the nerve fiber bundles, alteration of their thickness, alteration of their size), or alteration of the optic disc morphology (including but not limited to alteration of the optic nerve head, alteration of the physiological cup, alteration of the cup-disc ratio, alteration of the lamina cribrosa).
19. The method according to item 15, wherein said disease is diabetic retinopathy and the altered structure is an alteration of the retinal vasculature.
20. The method according to any one of item 1 to 12, wherein said altered structure is indicative of geographic atrophy, drusen, reticular pseudo-drusen, neovasculature and/or retinal pigment epithelium degeneration.
21. Use of an image of the eye of a subject obtained by transscleral optical imaging (TOI) for diagnosing, and/or prognosing a disease associated with an altered structure in the posterior segment of the eye, wherein the use comprises analyzing said image for an altered structure, wherein the altered structure is indicative of the presence and/or progression of the disease in said subject.
22. Use of an image of the eye of a subject obtained by TOI in treating a disease associated with an altered structure in the posterior eye segment, wherein the use comprises the steps of:
   a) analyzing said image for an altered structure, where the presence of an altered structure is indicative of the presence of the disease or the development of the disease in said subject; and
   b) administering to the subject identified as having or developing a disease according to step (a), an appropriate treatment for said disease.
23. The use according to item 21 or 22, wherein said altered structure is determined relative to a reference TOI image of said posterior segment obtained from a similarly situated subject known not to have the disease or known not to be at risk of developing the disease.
24. Use of a first and second image of the eye of a subject obtained by transscleral optical imaging (TOI) for
   (i) evaluating the therapeutic effect in a subject of a treatment for a disease associated with an altered structure in the poster segment of the eye, or
   (ii) determining a subject's compliance with a prescribed treatment for a disease associated with an altered structure in the posterior segment of the eye,
      said use comprising analyzing said first and second image,
(a) wherein said first image is to be obtained before said treatment or prior to second image;
(b) wherein said second image is to be obtained after treatment or subsequent to said first image;
   (c) wherein said analyzing the first and second image is an analysis and comparison of the structure of the posterior segment in (a) and (b);
      wherein the maintenance of or decrease in an altered structure between (a) and (b) is indicative of said treatment having therapeutic effect or said subject complying with said treatment.
25. The use according to item 24, wherein said altered structure is a structure altered relative to that determined from analysis of a TOI image of said posterior segment obtained from a similarly situated subject known not to have the disease or known not to be at risk of developing the disease.
26. The use according to item 24 wherein determination of the maintenance of the altered structure between (a) and (b) is
   (d) a determination where said altered structure is substantially unchanged between (a) and (b) of item 24; or
   (e) a determination where any further alteration or progression of said altered structure between (a) and (b) of item 24 is not as severe or advanced as that between a set of reference first and second TOI images of said posterior segment obtained from a similarly situated subject known to have the disease and which subject has not been treated for said disease, wherein the reference first and second images were obtained or are to be obtained at the same interval as the first and second images of (a) and (b) of item 24.
27. The use according to any one of items 24 to 26, wherein said second image is to be obtained at least 2 days and no more than 730 days subsequent to said first image.
28. The use according to any one of items 21 to 27, wherein said disease associated with an altered structure in the posterior segment of the eye is uveitis, glaucoma, macular edema, macular hole, macular pucker, diabetic macular edema, diabetic retinopathy, diabetic eye diseases, retinopathy, age-related macular degeneration (AMD), wet AMD, dry AMD, early AMD, intermediate AMD, central serous chorioretinopathy, scleritis, optic nerve degeneration, geographic atrophy, choroidal disease, ocular sarcoidosis, optic neuritis, choroidal neovascularization, retinitis pigmentosa, retinal tears, Stargardt disease, ocular cancer, retinitis, corneal ulcers, cataract, infection with a virus (such as cytomegalovirus, herpes simplex, herpes zoster), infection with fungi (such as histoplasmosis), parasitic infection (such as toxoplasmosis, toxocariasis), bacterial infection (such as tuberculosis, syphilis), sarcoidosis, retinal vein occlusion, central retinal vein occlusion, branch retinal vein occlusion, retinal vascular disease, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, iridocyclitis diabetic retinopathy, retinopathy of prematurity (ROP), ischemic vasculopathies, inherited retinal dystrophies, retinal detachment, aberrant angiogenesis, retinal angiomatous proliferation (RAP), intraretinal microvascular abnormalities, pre-retinal neovascularization, choroidal angiogenesis, choroidal vasculopathy stroke, hypertension, diabetes, cardiovascular disease, prematurity, and papilloedema.
29. The use according to any one of items 21 to 28, wherein the image of said posterior segment of the eye is an image of the choroid, the choriocapillaris, Bruch's membrane, retinochoroidal tissue, the neuroretinal tissue, the nerve fiber layer,, the retinal pigment epithelium (RPE), the photorepectors, the ganglion cell layer, the retinal vasculature, the subretinal space, the retina, the macula, the lamina cribrosa, the optic disc or the optic nerve.
30. The use according to any one of items 21 to 29, wherein said altered structure is an alteration in the tissue structure.
31. The use according to item 30, wherein the alteration in tissue structure is an alteration in cell pattern, cell density, cell size, cell distribution or cell reflectivity.
32. The use according to item 31, wherein said alteration in tissue structure is an alteration in tissue reflectivity that is an alteration in hyporeflective regions, hyperreflective regions, hyporeflective regions within a hyperreflective region, or any combination thereof.
33. The use according to any one of items 21 to 32, wherein said image is an image of the RPE.
34. The use according to any one of items 21 to 32, wherein said image is an image of the choriocapillaris.
35. The use according to any one of items 21 to 32, wherein the image is an image of the nerve fiber layer, optic disc and/or retinal vasculature.
36. The use according to item 33, wherein said disease is AMD and said altered structure is an alteration in RPE cell density, RPE cell size, hyperreflective regions, hyporeflective regions, and/or hyporeflective regions within a hyperreflective region.
37. The use according to item 33 or 34, wherein said disease is central serous chorioretinopathy and said altered structure is an alteration of the RPE and the choriocapillaris.
38. The use according to item 35, wherein said disease is glaucoma and said altered structure is an alteration of the nerve fiber layer (including but not limited to alteration of the orientation of the nerve fiber bundles, alteration of their thickness, alteration of their size), or alteration of the optic disc morphology (including but not limited to alteration of the optic nerve head, alteration of the physiological cup, alteration of the cup-disc ratio, alteration of the lamina cribrosa).
39. The use according to item 35, wherein said disease is diabetic retinopathy and the altered structure is an alteration of the retinal vasculature.
40. The use according to any one of items 21 to 32, wherein said altered structure is indicative of geographic atrophy, drusen, reticular pseudo-drusen, neovasculature and/or retinal pigment epithelium degeneration.

Accordingly, the present invention provides a highly accurate method to detect alterations in the structure of the posterior segment of the eye for the significantly improved diagnosis, prognosis, monitoring and treatment of diseases associated with said structural alterations.

According to one embodiment of the present invention, the invention provides a method of diagnosing, and/or prognosing a disease associated with an altered structure in the posterior segment of the eye, wherein the method comprises analyzing an image of the posterior segment of the eye obtained by transscleral optical imaging (TOI) for an altered structure, wherein the altered structure is indicative of the presence and/or progression of the disease in a subject.

In a further embodiment of the invention, the invention provides a method of treating a disease associated with an altered structure in the posterior eye segment, wherein the method comprises the steps of: a) analyzing an image of the posterior eye segment obtained by TOI for an altered structure, where the presence of an altered structure is indicative of the presence of the disease or the development of the disease in a subject; and b) administering to the subject identified as having or developing a disease according to step (a), an appropriate treatment for said disease.

In certain embodiments, the method of the present invention may refer to methods wherein said altered structure is determined relative to a reference that is a TOI image of said posterior segment obtained from a similarly situated subject known not to have the disease or known not to be at risk of developing the disease.

The methods described herein relate to the analysis of a TOI image of the eye for determination of an altered structure. The skilled person is aware of the anatomical structure of the posterior segment of the eye in the disease free condition (the anatomical structure in the normal eye) and, therefore can determine the presence or absence of an altered structure in the TOI image empirically, for example by methods including but not limited to, by visual inspection. However, the altered structure can also be determined by comparison to a reference. As it is used herein, the term “reference” refers to pre-determined or known structures of the posterior segment of the eye. Deviations in the image from the subject as compared from the reference determines an alteration in structure, which may, for example, indicate the presence of a disease state, the progression of a disease state or a predisposition to the development of a disease state. In certain embodiments, “reference” as used herein is a reference TOI image from a subject known not to have the disease or known not to be at risk for the development of the disease. In other embodiments, were it is desired to monitor progression of disease or compliance with a therapy, the reference may be a TOI image or set of TOI images obtained from a reference subject known to have the disease or known to be at a predisposition for developing the disease, which subject is untreated for such disease. In analysis according to the methods relative to reference to a set of images obtained from an untreated subject, the altered structures are considered to be maintained where the change in the altered structure(s) in the set of images from the analysis subject is not as progressed or not as advances as that in the reference images.

The altered structures of the posterior segment of the eye, whether determined empirically (i.e. without comparison to a reference) or relative to a reference, may be altered macroscopic or microscopic structures. Non-limiting examples of macroscopic structures include vasculature (such as, but not limited to retinal vasculature), wherein the altered structure may include (but is not limited to) altered size, altered vascular density, or altered vascular pattern. The altered structure can also be microscopic such as altered intracellular or extracellular changes. It is preferred that the determined altered structure is an altered cellular structure of a tissue of the posterior segment of the eye. Non-limiting alterations in cellular structure can include alterations in cell density, cell size, and/or cell pattern.

Microscopic alterations in structure need not be limited to alterations attributed to changes in any specific cell or groups of cells per se, but can be attributed to changes resulting from or dependent on alterations in their structure or phenotype. Such microscopic alterations in structure include alterations of hypo- or hyper-reflective regions. Accordingly, altered structure can include an alteration in hyporeflective regions, such as but not limited to alterations (increase or decrease) in the density, concentration, grouping, or pattern of hyporeflective regions. Altered structure can also include an alteration in hyperreflective regions, such as but not limited to alterations (increase or decrease) in the density, concentration, grouping, or pattern of hyperreflective regions. Alterations to structure can also include alterations to both hyper- and hypo-reflective regions as described in this paragraph or otherwise herein. Alterations to structure can also include alterations (appearance, disappearance, increase in concentration/density, or decreasing in concentration/density) of regions having both hyper- and hypo-reflective regions, e.g. hyporeflective regions within a hyper-reflective region (known in the art as hyporeflective regions surrounded by a hyperreflective halo.

Where the altered structure is determined relative to a reference, the reference need not necessarily be determined every time. A reference can be based, e.g., on a TOI image having been obtained from the subject being analyzed, but at an earlier point in time, including prior to therapeutic intervention. The reference image can additionally or alternately be based on a standard TOI image, e.g. an image obtained from an unrelated subject known not to have the relevant disease or known not to be at risk of developing the relevant disease. The reference can also be the result of standardization of a large number of images. In such cases both the standardization of the reference image or images and analysis of the subject image can be made by a machine learning tool, e.g. a computer having appropriate image analysis software.

It will be appreciated that the structure of the posterior segment of the eye, whether macro or microscopic, is dependent on a number of factors, for example the age and gender of the subject, whether they are subject to medical therapies (e.g. are being treated with therapeutic drugs which may or may not be related to the disease under analysis) and/or their lifestyle habits (e.g. whether they are smokers, consume alcohol, level of fitness, etc.). Accordingly, where the reference is a standard image, the reference image may be obtained from a similarly situated source or group of sources as the subject, e.g. a source or group of sources having similar physical characteristics as the subject and having similar lifestyle criteria. In view of the potential variation, average structural characteristics may be developed from a large number of sources known to not have the disease or known to not be at risk for having the disease for use as a reference.

The “TOI device” developed by the inventors of the present application, refers to a device for the ophthalmic illumination of the eye fundus using a light-delivering device with multiple light sources; where each light source is configured to be independently controllable and directed towards the sclera of the eye, providing transscleral oblique illumination of the eye fundus; an active eye aberration correcting system; and an imaging system configured to create multiple images of the eye fundus on multiple imaging sensors. The light transmitted through the sclera creates an oblique illumination of the posterior retina; this is then imaged using a transpupillary AO full-field camera system. The TOI device provides dark field imaging, high resolution imaging and large field of view (FOV) imaging.

The present inventors have found that the TOI device advantageously provides cellular-resolution label-free high-contrast images of the posterior eye segment, in particular the retinal layers over a large FOV without the drawback of a long exposure time. Oblique illumination, including transscleral or transpalpebral (e.g. transscleral flood illumination) of the retina as used in TOI greatly increases the signal-to-noise ratio (SNR) of many retinal structures as compared to transpupillary illumination.

The TOI device as used herein uses an aberration correction method. The correction of the optical aberrations is performed in real-time with but not limited to an adaptive optics closed-loop comprising a transpupil probing light source, a wavefront sensor and a wavefront corrector able to spatially shape the wavefront of the light making a front-facing image. The TOI device combines transpupil or transpupillary illumination and transscleral illumination to benefit from the advantages of the two types of illumination.

The term “transscleral” means across the sclera, or white, of the eye. The term “sclera”, as used herein, refers to the white of the eye which is the opaque, fibrous, protective, outer layer of the human eye containing mainly collagen and some elastic fiber. The sclera is a connective tissue made mostly of white collagen fibers. It underlies the choroid posteriorly and continues anteriorly where it becomes transparent over the iris and pupil and is referred to as the cornea.

The term “diagnosis”, as used herein, means confirmation of the presence or characteristics of a pathological condition. With regard to the present invention, diagnosis means confirmation of the presence of an altered structure of the posterior segment of the eye. The altered structure may refer to alterations in the anterior hyaloid membrane, vitreous humor, retina, choroid, and/or optic nerve.

The term “prognosis”, as used herein, refers to the prediction of the probable development or outcome of a disease or the likelihood of recovery from a disease. As will be understood by those skilled in the art, the prediction, although preferred to be, need not be correct for 100% of the subjects to be diagnosed or evaluated. The term, however, requires that a statistically significant portion of subjects can be identified as having an increased probability of having a given outcome.

The term “treatment” of a disorder or disease, as used herein, is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease). The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “posterior eye segment”, or grammatical variations thereof, refers to the portion of the eye that is behind the lens or the ora serata. This portion is comprised of the back ⅔ of the eye that includes the anterior hyaloid membrane and all of the optical structures behind it: the vitreous humor, retina, choroid, and optic nerve. “Posterior eye segment diseases” or “diseases associated with an altered structure in the posterior eye segment”, or grammatical variations thereof, as used herein, refer to diseases affecting the posterior segment of the eye. Posterior eye segment diseases include, but are not limited to uveitis, glaucoma, macular edema, macular hole, macular pucker, diabetic macular edema, diabetic retinopathy, diabetic eye diseases, retinopathy, age-related macular degeneration (AMD), wet AMD, dry AMD, early AMD, intermediate AMD, central serous chorioretinopathy, scleritis, optic nerve degeneration, geographic atrophy, choroidal disease, ocular sarcoidosis, optic neuritis, choroidal neovascularization, retinitis pigmentosa, retinal tears, Stargardt disease, ocular cancer, retinitis, corneal ulcers, cataract, infection with a virus (such as cytomegalovirus, herpes simplex, herpes zoster), infection with fungi (such as histoplasmosis), parasitic infection (such as toxoplasmosis, toxocariasis), bacterial infection (such as tuberculosis, syphilis), sarcoidosis, retinal vein occlusion, central retinal vein occlusion, branch retinal vein occlusion, retinal vascular disease, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, iridocyclitis diabetic retinopathy, retinopathy of prematurity (ROP), ischemic vasculopathies, inherited retinal dystrophies, retinal detachment, aberrant angiogenesis, retinal angiomatous proliferation (RAP), intraretinal microvascular abnormalities, pre-retinal neovascularization, choroidal angiogenesis, choroidal vasculopathy stroke, hypertension, diabetes, cardiovascular disease,, prematurity, and papilloedema.

As used herein, the term “subject” refers to a mammal including a non-primate (e.g., a camel, donkey, zebra, cow, pig, horse, cat, dog, rat, and mouse) and a primate (e.g., a monkey, chimpanzee, and a human). In certain embodiments, the subject suffers or is susceptible to suffer from a disease characterized by a alteration of the posterior eye segment and is preferably human.

In certain embodiments of the present invention, the method relates to a method for (i) evaluating the therapeutic effect in a subject of a treatment for a disease associated with an altered structure in the poster segment of the eye, or (ii) determining a subject's compliance with a prescribed treatment for a disease associated with an altered structure in the posterior segment of the eye, said method comprising analyzing a first and second image of the posterior segment of the eye of said subject obtained by TOI, (a) wherein said first image is to be obtained before said treatment or prior to second image; (b) wherein said second image is to be obtained after treatment or subsequent to said first image; (c) wherein the analyzing the first and second image is an analysis and comparison of the structure of the posterior segment in (a) and (b); wherein the maintenance of or decrease in an altered structure between (a) and (b) is indicative of said treatment having therapeutic effect or said subject complying with said treatment.

Moreover, the methods of the present invention may relate to methods wherein the therapeutic effect in a subject or compliance of the subject with a prescribed treatment is determined by analysis of the altered structure relative to that determined from analysis of a TOI image of said posterior segment obtained from a similarly situated subject known not to have the disease or known not to be at risk of developing the disease.

In a further embodiment of the invention, the method relates to a method wherein determination of the maintenance of the altered structure between (a) and (b) is (d) a determination where said altered structure is substantially unchanged between (a) and (b) of the aforementioned method; or (e) a determination where any further alteration or progression of said altered structure between (a) and (b) of the aforementioned method is not as severe or advanced as that between a set of reference first and second TOI images of said posterior segment obtained from a similarly situated subject known to have the disease and which subject has not been treated for said disease, wherein the reference first and second images were obtained or are to be obtained at the same interval as the first and second images of (a) and (b) of the aforementioned method.

In certain embodiments of the invention, the method relates to methods wherein the second image is to be obtained at least 2 days and no more than 730 days subsequent to said first image.

The inventors of the present invention found that the evaluation of the therapeutic effect or compliance of a subject with a given therapy could be accurately predicted by analyzing first and second images of the posterior eye segment when said images were obtained at least 2 days and no more than 730 days apart. The superior images obtained by the TOI device thus provide a means and method for early detection, intervention and fast analysis of the prognosis of a disease state. This is essential in improving treatment choice, treatment compliance and disease outcome.

In one embodiment of the invention, the method relates to methods wherein said disease associated with an altered structure in the posterior segment of the eye is uveitis, glaucoma, macular edema, macular hole, macular pucker, diabetic macular edema, diabetic retinopathy, diabetic eye diseases, retinopathy, age-related macular degeneration (AMD), wet AMD, dry AMD, early AMD, intermediate AMD, central serous chorioretinopathy, scleritis, optic nerve degeneration, geographic atrophy, choroidal disease, ocular sarcoidosis, optic neuritis, choroidal neovascularization, retinitis pigmentosa, retinal tears, Stargardt disease, ocular cancer, retinitis, corneal ulcers, cataract, infection with a virus (such as cytomegalovirus, herpes simplex, herpes zoster), infection with fungi (such as histoplasmosis), parasitic infection (such as toxoplasmosis, toxocariasis), bacterial infection (such as tuberculosis, syphilis), sarcoidosis, retinal vein occlusion, central retinal vein occlusion, branch retinal vein occlusion, retinal vascular disease, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, iridocyclitis diabetic retinopathy, retinopathy of prematurity (ROP), ischemic vasculopathies, inherited retinal dystrophies, retinal detachment, aberrant angiogenesis, retinal angiomatous proliferation (RAP), intraretinal microvascular abnormalities, pre-retinal neovascularization, choroidal angiogenesis, choroidal vasculopathy stroke, hypertension, diabetes, cardiovascular disease, prematurity, and papilloedema.

In a preferred embodiments of the invention, the method relates to methods wherein the image of said posterior segment of the eye is an image of the choroid, the choriocapillaris, Bruch's membrane, retinochoroidal tissue, the neuroretinal tissue, the nerve fiber layer, the retinal pigment epithelium (RPE), the photorepectors, the ganglion cell layer, the retinal vasculature, the subretinal space, the retina, the macula, the lamina cribrosa, the optic disc or the optic nerve.

In one preferred embodiment of the invention, the method relates to methods wherein said altered structure is an alteration in the tissue structure.

In certain embodiments of the invention, the method relates to methods wherein the alteration in tissue structure is an alteration in cell pattern, cell density, cell size, cell distribution or cell reflectivity.

The inventors found that, in particular, the examination of the cellular structure of the posterior eye segment for alterations in the structure provides an accurate means for the early detection of a disease and monitoring of the therapeutic effect of a given treatment for said disease.

By analysing the cellular structure to identify alterations in cell pattern, density, size, distribution and reflectivity, the inventors found that these particular features could be used to accurately assess disease state.

In a further preferred embodiment of the invention, the method relates to methods wherein said alteration in tissue structure is an alteration in tissue reflectivity that is an alteration in hyporeflective regions, hyperreflective regions, hyporeflective regions within a hyperreflective region, or any combination thereof.

Alterations in cell reflectivity, in particular alterations in hyporeflective regions, hyperreflective regions and/or hyporeflective regions within a hyperreflective region could be used to provide crucial information on the morphological changes occurring in the tissue structure. Therefore, the examination of these features is particularly advantageous for the assessment of the posterior eye segment.

In one preferred embodiment of the invention, the method relates to methods wherein said image is an image of the RPE.

The inventors of the present invention surprisingly found that by analyzing an image of the RPE at the cellular level, certain cellular morphological characteristics could be identified as early indicators for the onset of a disease. The miniscule changes that occur in the RPE layer, namely changes in cell density, number of neighbors, eccentricity and form factor cannot be examined by methods currently available in the art. This is due to the low contrast between neighboring cells, motion artefacts, retinal layer non-linearity, and difficulties with the image's focal point identification. The method of the present invention therefore provides specific indicators which can be used for the early detection and prognosis of a disease.

In a another preferred embodiment of the invention, the image is an image of the choriocapillaris.

In yet another embodiment of the invention, the image is an image of the nerve fiber layer, optic disc and/or retinal vasculature.

In a further embodiment of the invention, the method relates to methods wherein said disease is AMD and said altered structure is an alteration in RPE cell density, RPE cell size, hyperreflective regions, hyporeflective regions, and/or hyporeflective regions within a hyperreflective region.

The transition from intermediate AMD to advanced AMD can be delayed and possibly prevented by taking a specific high-dose formulation of antioxidants and zinc. Research has shown that a daily intake of supplements, including: vitamin C (500 milligrams); vitamin E 400 IU; beta-carotene (15 milligrams); zinc (as zinc oxide) (80 milligrams); and copper (as cupric oxide) (2 milligrams), reduced the risk of patients advancing from intermediate AMD to advanced AMD by 25%, and reduced the risk of vision loss by 19%. (www.amd.org). Currently there are four treatments approved by the FDA for wet AMD: laser surgery, photodynamic therapy (PDT), and the drugs Macugen® pegaptanib sodium and Lucentis™ ranibizumab intravitreal injections. Laser, PDT and pegaptanib may slow the rate of vision decline and/or stop vision loss. Pegaptanib (Macugen®, Eyetech Pharmaceuticals Inc. and Pfizer Inc.), is approved for treatment of wet AMD is a pegylated oligonucleotide aptamer targeting VEGF. Ranibizumab (Lucentis™, Genentech/Novartis), an antibody fragment targeting VEGF, has recently been approved by FDA for the treatment of wet AMD. Laser surgery attempts to destroy the fragile, leaky blood vessels using a high energy beam of light.

In a further embodiment of the invention, the method relates to methods wherein said disease is central serous chorioretinopathy and said altered structure is an alteration of the RPE and the choriocapillaris.

CSCR is usually a self-limiting disease with spontaneous resolution within 3-4 months with overall good visual outcome. However, recurrences are seen in up to 50% of patients within the first year. Current treatment approaches include photodynamic therapy, oral aldosterone antagonism and subthreshold multifocal laser. There has also been further investigation into a number of new treatments including antivascular endothelial growth factor treatment.

In one embodiment of the invention, the method relates to methods wherein said disease is glaucoma and said altered structure is an alteration of the nerve fiber layer (including but not limited to alteration of the orientation of the nerve fiber bundles, alteration of their thickness, alteration of their size), or alteration of the optic disc morphology (including but not limited to alteration of the optic nerve head, alteration of the physiological cup, alteration of the cup-disc ratio, alteration of the lamina cribrosa).

Glaucoma has been simply defined as the process of ocular tissue destruction caused by a sustained elevation of the Intra Ocular Pressure (IOP) above its normal physiological limits. Although several etiologies may be involved in the glaucoma complex, an absolute determinant in therapy selection is the amount of primary and/or induced change in pressure within the iridocorneal angle. Current therapies include medications or surgeries aimed at lowering this pressure, although the pathophysiological mechanisms by which elevated IOP leads to neuronal damage in glaucoma are unknown. Medical suppression of an elevated IOP can be attempted using four types of drugs: (1) the aqueous humor formation suppressors (such as carbonic anhydrase inhibitors, beta-adrenergic blocking agents, and alpha2-adrenoreceptor agonists); (2) miotics (such as parasympathomimetics, including cholinergics and anticholinesterase inhibitors); (3) uveoscleral outflow enhancers; and (4) hyperosmotic agents (that produce an osmotic pressure gradient across the blood/aqueous barrier within the cilliary epithelium). A fifth category of drugs, neuroprotection agents, is emerging as an important addition to medical therapy, including, for example, NOS inhibitors, excitatory amino acid antagonists, glutamate receptor antagonists, apoptosis inhibitors, and calcium channel blockers.

In one embodiment of the invention, the invention relates to methods wherein said disease is diabetic retinopathy and the altered structure is an alteration of the retinal vasculature.

In certain embodiments of the invention, the method relates to methods wherein said altered structure is indicative of geographic atrophy, drusen, reticular pseudo-drusen, neovasculature and/or retinal pigment epithelium degeneration.

The term “geographic atrophy”, as used herein, refers to advanced dry AMD. Geographic atrophy is characterized by an “island” of atrophied photoreceptors cells. It is believed that the alternative complement pathway may play a role in the pathogenesis of AMD.

The term “drusen”, as used herein, refers to yellowish deposits located deep to the RPE in the inner aspect of Bruch's membrane.

The method of the present invention also provides the use of an image of the eye of a subject obtained by TOI in treating a disease associated with an altered structure in the posterior eye segment, wherein the use comprises the steps of: a) analyzing said image for an altered structure, where the presence of an altered structure is indicative of the presence of the disease or the development of the disease in said subject; and b) administering to the subject identified as having or developing a disease according to step (a), an appropriate treatment for said disease.

In certain embodiments of the invention, the use of the present invention relates to the use wherein said altered structure is determined relative to a reference TOI image of said posterior segment obtained from a similarly situated subject known not to have the disease or known not to be at risk of developing the disease.

In another embodiment of the invention, the invention provides for the use of a first and second image of the eye of a subject obtained by transscleral optical imaging (TOI) for (i) evaluating the therapeutic effect in a subject of a treatment for a disease associated with an altered structure in the poster segment of the eye, or (ii) determining a subject's compliance with a prescribed treatment for a disease associated with an altered structure in the posterior segment of the eye, said use comprising analyzing said first and second image, (a) wherein said first image is to be obtained before said treatment or prior to second image; (b) wherein said second image is to be obtained after treatment or subsequent to said first image; (c) wherein said analyzing the first and second image is an analysis and comparison of the structure of the posterior segment in (a) and (b); wherein the maintenance of or decrease in an altered structure between (a) and (b) is indicative of said treatment having therapeutic effect or said subject complying with said treatment.

Furthermore, in one embodiment, the use of a first and second image, wherein said altered structure is a structure altered relative to that determined from analysis of a TOI image of said posterior segment obtained from a similarly situated subject known not to have the disease or known not to be at risk of developing the disease.

In another embodiment of the invention, the aforementioned use relates to the use wherein determination of the maintenance of the altered structure between (a) and (b) is (d) a determination where said altered structure is substantially unchanged between (a) and (b) of the aforementioned use; or (e) a determination where any further alteration or progression of said altered structure between (a) and (b) of the aforementioned use is not as severe or advanced as that between a set of reference first and second TOI images of said posterior segment obtained from a similarly situated subject known to have the disease and which subject has not been treated for said disease, wherein the reference first and second images were obtained or are to be obtained at the same interval as the first and second images of (a) and (b) of the aforementioned use.

In a preferred embodiment of the invention, the use of the invention relates to uses wherein said disease associated with an altered structure in the posterior segment of the eye is uveitis, glaucoma, macular edema, macular hole, macular pucker, diabetic macular edema, diabetic retinopathy, diabetic eye diseases, retinopathy, age-related macular degeneration (AMD), wet AMD, dry AMD, early AMD, intermediate AMD, central serous chorioretinopathy, scleritis, optic nerve degeneration, geographic atrophy, choroidal disease, ocular sarcoidosis, optic neuritis, choroidal neovascularization, retinitis pigmentosa, retinal tears, Stargardt disease, ocular cancer, retinitis, corneal ulcers, cataract, infection with a virus (such as cytomegalovirus, herpes simplex, herpes zoster), infection with fungi (such as histoplasmosis), parasitic infection (such as toxoplasmosis, toxocariasis), bacterial infection (such as tuberculosis, syphilis), sarcoidosis, retinal vein occlusion, central retinal vein occlusion, branch retinal vein occlusion, retinal vascular disease, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, iridocyclitis diabetic retinopathy, retinopathy of prematurity (ROP), ischemic vasculopathies, inherited retinal dystrophies, retinal detachment, aberrant angiogenesis, retinal angiomatous proliferation (RAP), intraretinal microvascular abnormalities, pre-retinal neovascularization, choroidal angiogenesis, choroidal vasculopathy stroke, hypertension, diabetes, cardiovascular disease, prematurity, and papilloedema.

In a certain embodiments of the invention, the invention relates to the use wherein the image of said posterior segment of the eye is an image of the choroid, the choriocapillaris, Bruch's membrane, retinochoroidal tissue, the neuroretinal tissue, the nerve fiber layer,, the retinal pigment epithelium (RPE), the photorepectors, the ganglion cell layer, the retinal vasculature, the subretinal space, the retina, the macula, the lamina cribrosa, the optic disc or the optic nerve.

In certain embodiments of the invention, the invention relates to the use wherein said altered structure is an alteration in the tissue structure.

In certain embodiments of the invention, the invention relates to the use wherein the alteration in tissue structure is an alteration in cell pattern, cell density, cell size, cell distribution or cell reflectivity.

In certain embodiments of the invention, the invention relates to the use wherein the alteration in tissue structure is an alteration in cell pattern, cell density, cell size, cell distribution or cell reflectivity.

In certain embodiments of the invention, the invention relates to the use wherein said alteration in tissue structure is an alteration in tissue reflectivity that is an alteration in hyporeflective regions, hyperreflective regions, hyporeflective regions within a hyperreflective region, or any combination thereof.

In a preferred embodiment of the invention, the invention relates to the use of an image wherein said image is an image of the RPE.

In yet another preferred embodiment of the invention, the invention relates to the use of an image wherein said image is an image of the choriocapillaris.

In certain embodiments of the invention, the invention relates to the use of an image wherein the image is an image of the nerve fiber layer, optic disc and/or retinal vasculature.

In certain embodiments of the invention, the invention relates to the use wherein said disease is AMD and said altered structure is an alteration in RPE cell density, RPE cell size, hyperreflective regions, hyporeflective regions, and/or hyporeflective regions within a hyperreflective region.

In certain embodiments of the invention, the invention relates to the use wherein said disease is central serous chorioretinopathy and said altered structure is an alteration of the RPE and the choriocapillaris.

In certain embodiments of the invention, the invention relates to the use wherein said disease is glaucoma and said altered structure is an alteration of the nerve fiber layer (including but not limited to alteration of the orientation of the nerve fiber bundles, alteration of their thickness, alteration of their size), or alteration of the optic disc morphology (including but not limited to alteration of the optic nerve head, alteration of the physiological cup, alteration of the cup-disc ratio, alteration of the lamina cribrosa).

In certain embodiments of the invention, the invention relates to the use wherein said disease is diabetic retinopathy and the altered structure is an alteration of the retinal vasculature.

In certain embodiments of the invention, the invention relates to the use wherein said altered structure is indicative of geographic atrophy, drusen, reticular pseudo-drusen, neovasculature and/or retinal pigment epithelium degeneration.

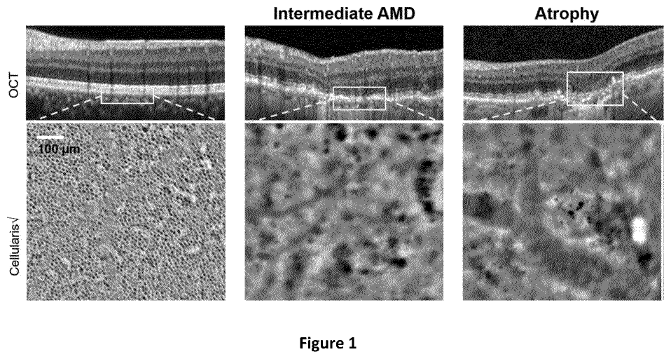

FIG. 1 Examples of images seen in different degrees of non-neovascular AMD and comparison with SD OCT.

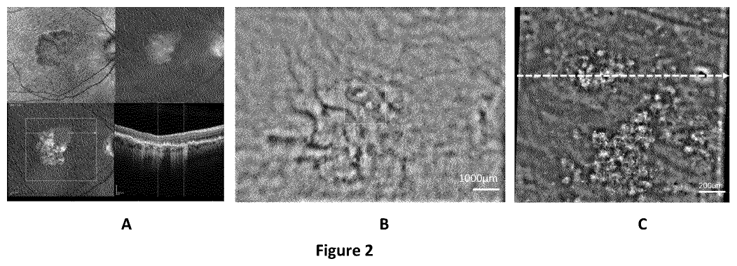

FIG. 2 Comparison between images taken with SD OCT, Autofluorescence and color with the results found in TOI Images of an AMD patient with focal geographical atrophy. The TOI image shows a reticular choroidal pattern with dark spots (or hyporeflective regions) of variable size correlating with degenerative remnants of RPE.

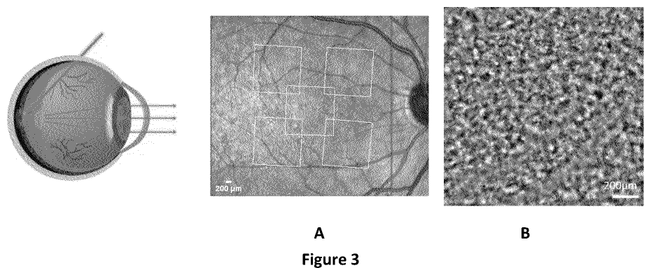

FIG. 3 A) 5°×5° zones acquired within the fovea and the macula (5.4° eccentricity). B) in vivo RPE image (temporal, inferior to fovea) of the contralateral normal eye of CSCR patient. The absorption of infrared (830 nm) incident light by the melanin in RPE leads to a hyporeflective core region on TOI.

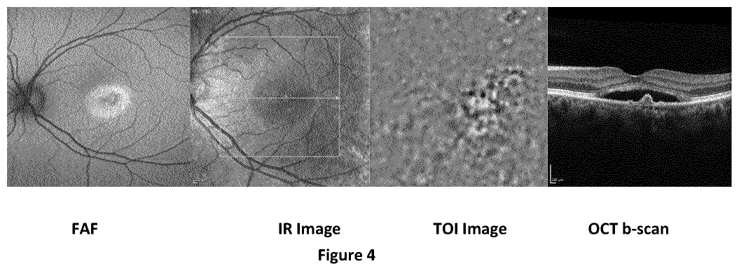

FIG. 4 TOI at the fovea (middle right) in a case of neurosensory detachment Low resolution image with hyper-reflective dots with pigment epithelial detachment and hyperreflective contents on OCT (extreme right). FAF (extreme left) shows hyper autofluorescence while IR (middle right) shows a hyporeflective zone in the area of detachment with central hyperreflectivity.

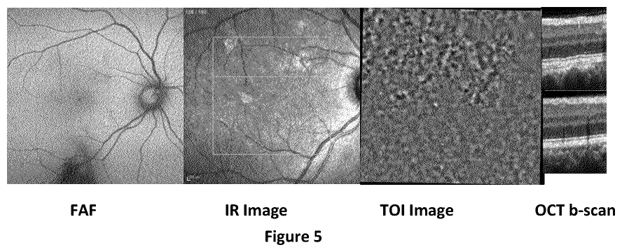

FIG. 5 TOI depicting an altered RPE mosaic in the upper half (center) with corresponding inner choroid change on OCT (right) while the RPE anatomy is maintained in the lower half (center) with a normal corresponding OCT (left). These areas showed changes on infrared imaging confirming the anatomical location of pathology.

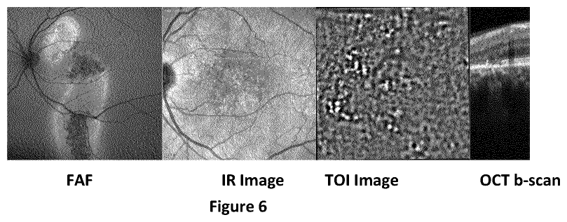

FIG. 6 FAF (extreme left) shows a descending tract with hyper and hypo AF while IR (middle left) shows areas of hyperreflectivity. TOI (middle right) shows multiple hyperreflective foci corresponding hyperreflective deposits on OCT (extreme right, between vertical lines). Hyperreflective dots represent areas of increase reflectance secondary to the disease process.

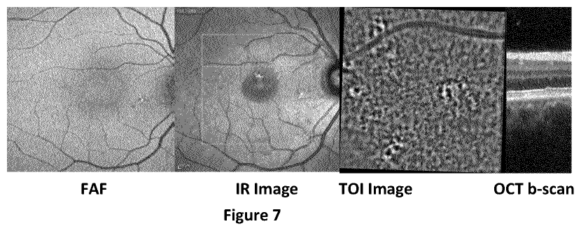

FIG. 7 Loss of central hyporeflective regions of FAF (left) with a central hyporeflective region with hyperreflectivity on IR (center) secondary to neurosensory detachment is noted. TOI (middle right) demonstrates multiple hyporeflective regions (foci) suggestive of dark dots with inner choroid change in the corresponding OCT.

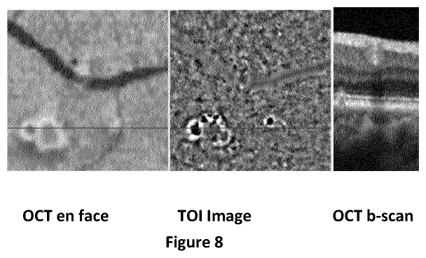

FIG. 8 AMD patient with drusen. The confocal TOI image (middle) shows the drusen as dark center and white halo (hyporeflective region within hyperreflective region). Not all such TOI appearance is visible neither on OCT en face nor b-scan. A retinal vessel crosses the image, hiding the deeper structures.

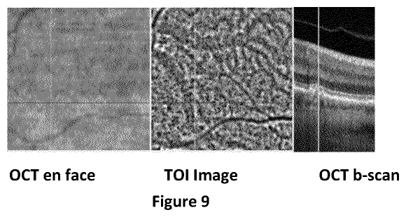

FIG. 9 AMD patient with few intermediate drusen. The confocal TOI image shows a large number of small dark spots, partially with white halo (hyporeflective regions within hyperreflective region), most of them not clearly identifiable on OCT. However, there are numerous small RPE changes on OCT b-scans, which might translate deposits visible on TOI. Background color on TOI shows the choroidal and choriocapillaris vessels.

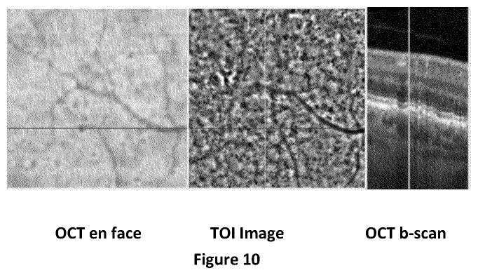

FIG. 10 AMD patient with drusen, well visibles on the OCT en face image. Confocal TOI shows a large number of irregularly distributed dark spots, partially with white halo (hyporeflective regions within hyperreflective region). Some of them are arranged similarly to the typical hexagonal pattern of normal RPE cells (yellow circle). In addition, both choroidal and retinal vessels are visible.

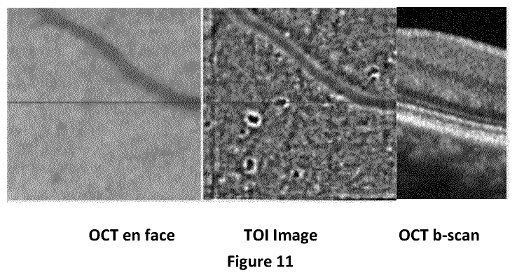

FIG. 11 AMD patient with small drusen. Confocal TOI shows darks pots with white halo spots (hyporeflective regions within hyperreflective regions) corresponding to the drusen. More dark spots (hyporeflective regions) are visible on TOI, without correspondence to the OCT image, probably too small to be visible on OCT.

### GENERAL METHODS AND MATERIALS

### TOI image acquisition

TOI relies on high-angle oblique illumination of the retina, combined with a flood illumination adaptive optics fundus camera, to enhance cell contrast and correct for ocular aberrations. Transscleral illumination of the retina was performed using two near-infrared light-emitting diodes located on the nasal and temporal side of the eye. The acquired images encompass a field-of-view of at least 4°×4°.

A single TOI-obtained RPE layer image is characterized by a low signal-to-noise ratio (SNR). Therefore, prior to image analysis, the SNR is increased by acquiring several raw images then registered, and averaged into a single TOI image. The final TOI-RPE images were exported with a digital sampling between 0.73 μm and 1.5 μm per pixel.

### Image Processing

The highly automated TOI-obtained RPE image processing and analysis is divided into four stages. First, the images are normalized in terms of contrast/attenuation, unevenness of the RPE layer and noise, and any out-of-focus (OoF) areas are discarded. Second, the shadow of retinal vasculature present in the innermost (vitreal-most) retinal layers is detected and removed from the final image. Third, cells or structures of interest are individually detected and segmented. Finally, the fourth and last step consists of characterizing the layer of interest in general and single structures.

### Image Filtering and Normalisation

In order to adjust for the unevenness of the layer of interest background, a background removale is applied. It may include for instance, but not limited to flat-field correction with a two-dimensional Gaussian smoothing kernel.

For example, for RPE layer, to prevent the filtering out of essential RPE morphology, both in the spatial and frequency domain, the filter sizes, thresholds, and values implemented throughout the image processing and analysis methodology, were obtained experimentally and based on previously published literature in the assessment of ex vivo and in vivo morphology of RPE cells.

### Detection and Removal of Blood Vessels

As a example, the detection of blood vessels is performed by using the following method. Other methods based on machine learning may be applied.

The detection of blood vessels is performed by using the four previously obtained images (/i|_{IpΓ}. Gauss, deH, and Dist). Each image is subjected to Subroutine A (SubA). SubA begins with square-shaping the image, and its quadtree decomposition (QuaD) returns a sparse matrix subsequently reconstructed as a block-map. The QuaD threshold is applied at 3*SD of the image. QuaD is a common methodology in several fields, including image processing, being used from multiresolution decomposition and analysis, to compression and machine learning. Application of QuaD for RPE cells seg-mentation is a novel approach developed specifically for this project. The QuaD square blocks of >8 pixels and <10% of the original image size are included in the subsequent image processing. After inverting (image complement), the obtained square blocks maps, small and interconnected structures at their external borders are discarded using morphological filtering (erosion with a discoid element of 4-pixel radius) followed by dilation with the same discoid element. Finally, the last step of SubA is reshaping of the resultant mask to the original's image size.

The OoF mask obtained during image filtering and normalization stage is summed with the binary mean of SubA (Bhpf)-SubA (Causs), SubA (deH), and SubA (Dist), forming the vessel-OoF mask (VOoF). VOoF mask is used to eliminate the infravascular RPE cells from further image processing.

### Cell Detection

Cell center detection is based on the method proposed by Khamidakh et al. (Ann Biomed Eng. 2016; 44:3408-20), henceforth named Subroutine B (SubB). In case the distance between adjacent cellular centres is <10 pixels, the individual cells are detected as the same cell. We applied SubB to Z?hpf-to the contrast-limited adaptive histogram equalised Z?hpf-and to the highpass filtered (| of the original image sized kernel)/i|_{IpΓ}. One more time, cellular centers within <10 pixels are fused. Finally, cellular centers in the distance of <10 pixels from the image border are removed to prevent the inclusion of non-fully-imaged cells in the image analysis.

### Cell Membrane Segmentation

### Method Example 1

Detection of the cellular membrane can be performed using local minima detection for the detection of the centers of hyporeflective or hyperreflective regions. Then, a region growing algorithm is applied to define the regions of interest.

### Method Example 2

Detection of the cellular membrane at the single-cell level begins with convolving Z?hpf with a discoid structuring element (radius of 4 pixels). The resultant blurring of the image removes any possible local salt-and-pepper noise that might occur during the transformation from the Fourier to the spatial domain. Then, the image is convolved with a star-shaped mask (size 7 pixels). The convolution enhances local vertical, horizontal, and diagonal edges in the image. The final hlter is a 7×7-pixel Mexican hat. With these three filtering stages followed by zero-crossing in the spatial domain, a binary mask representing the cellular membrane is developed. Finally, the mask is skeletonized and cleaned from sporadic branches, while single pixels are discarded. The inverted mask is convolved with a discoid structuring element (radius of 4 pixels) and re-inverted. Such a procedure improves the separation of the cells and prevents their possible overlapping.

### Data Analysis

### Example Considering RPE Cells

Cells with area or center overlapping with the VOoF mask were discarded from the analysis of cellular characteristics.

Using the previously created cellular masks and the original TOI-obtained image, morphological and neighborhood characteristics of individual RPE cells were assessed. MATFAB regionprops function was used to obtain basic morphological characteristics of RPE cells (area, centroid and weighted centroid, eccentricity, solidity, intensity, and circularity). In addition, assessed characteristics included the CV of RPE cellular membrane (CMDcv), number of neighboring cells and the cellular density of the RPE layer. To decrease the possible risk of assessment bias, RPE cells immediately adjacent to the VOof mask were discarded from the number of neighbours' evaluation. A descriptive analysis was conducted for each image.

The normality of variables was assessed with the Shapiro-Wilk's test (p>0.10) and histogram skewness (skewness −0.5-0.5).

The image processing pipeline and the underlying algorithms were developed and tested, as well as data management, on a DEFF workstation (DEFF XPS 13 9380, Windows 10, 64 bits, 2 1.80 GHz, 16.0 GB RAM) equipped with the MATFAB (version R2019, with Bioinformatics Toolbox™, Financial Toolbox™ and Statistics and Machine Teaming Toolbox™). Image registration was performed with ImageJ 1.52 with a modified macro with the plugins TurboReg and Template Matching. For boxplots generation and statistical analysis, we used R studio 1.2.1335 with gmodels, el071, readxl, and xlsx packages.

### TOI in Central Serous Chorioretinopathy (CSCR)

Patients with a clinical diagnostic of CSCR, clear optic media and good fixation were recruited. For each patient, BCVA, refractive error spherical equivalent, and axial length measurements, fundus autofluorescence (FAF), infrared imaging (IR) and SD-optical coherence tomography (OCT) were performed in both eyes.

TOI was successfully performed on 12 patients (21 CSCR eyes and 1 normal contralateral eye) with a mean age 43.3±4 years. The RPE structure or mosaic appeared as a fine network of cells with a hyporeflective (core) regions within hyperreflective regions (borders) (FIG. 3B)

Eyes with active CSCR (N=7) showed poor resolution in areas with retinal detachment (FIG. 4) on TOI. The presence of subretinal fluid in the pathway of light scattered from the RPE resulted in reduced resolution of RPE monolayer imaging.

Resolved CSCR eyes (N=14) showed an altered pattern of RPE mosaic on TOI (FIG. 5). Alterations in the scattering surface (RPE and choroid) secondary to CSCR leads to altered mosaic patterns on TOI.

Hyperreflective dots on TOI were observed in all 21 eyes corresponding to hyperreflective outer retinal deposits (FIG. 4) or RPE detachments with hyperreflective content on OCT (FIG. 6).

TOI images also revealed dark dots (hypo-reflective foci) in 19 out of 21 eyes. They were associated with normal surrounding RPE, hyperreflective structures or zones (FIG. 7).

### TOI in Non-Neovascular Age Related Macular Degeneration

Patients with non-neovascular AMD, clear optic media and good fixation were recruited. For each patient, SD-OCT, autofluorescence, color and infrared fundus imaging and TOI were performed in one or both eyes. A comparison was made of images obtained by TOI against conventional imaging including OCT en face and OCT b-scan (FIGS. 8 to 11).

Included were 31 eyes of 25 AMD patients (mean age 71.8 years, 56% females). Prominent features on the TOI images were the reticular or cell pattern of the choriocapillaris, and the presence of additional irregularly distributed hyporeflective regions. These hyporeflective regions, with size varying from 1 time to approximately 15 times the mean RPE cell size, were located on the low reflectance areas of the choriocapillaris, and particularly present in the border area around atrophic zones and in areas of visible RPE changes. A subgroup of these cells, showing a bright halo around them (hyporeflective regions within a hyperreflective region), correlated well with drusenoid deformation of the RPE line on SD-OCT.

TOI performed in 25 AMD patients revealed different patterns of altered tissue in the RPE layer. TOI is therefore an important tool in the evaluation of retinal diseases such as AMD.

1. A method of diagnosing, and/or prognosing a disease associated with an altered structure in the posterior segment of the eye, wherein the method comprises analyzing an image of the posterior segment of the eye obtained by transscleral optical imaging (TOI) for an altered structure, wherein the altered structure is indicative of the presence and/or progression of the disease in a subject.

2. A method of treating a disease associated with an altered structure in the posterior eye segment, wherein the method comprises the steps of:
a) analyzing an image of the posterior eye segment obtained by TOI for an altered structure, where the presence of an altered structure is indicative of the presence of the disease or the development of the disease in a subject; and
b) administering to the subject identified as having or developing a disease according to step (a), an appropriate treatment for said disease.

3. The method according to claim 1, wherein said altered structure is determined relative to a reference that is a TOI image of said posterior segment obtained from a similarly situated subject known not to have the disease or known not to be at risk of developing the disease.

4. A method for
(i) evaluating the therapeutic effect in a subject of a treatment for a disease associated with an altered structure in the poster segment of the eye, or
(ii) determining a subject's compliance with a prescribed treatment for a disease associated with an altered structure in the posterior segment of the eye,
said method comprising analyzing a first and second image of the posterior segment of the eye of said subject obtained by TOI,
(a) wherein said first image is to be obtained before said treatment or prior to second image;
(b) wherein said second image is to be obtained after treatment or subsequent to said first image;
(c) wherein the analyzing the first and second image is an analysis and comparison of the structure of the posterior segment in (a) and (b);
wherein the maintenance of or decrease in an altered structure between (a) and (b) is indicative of said treatment having therapeutic effect or said subject complying with said treatment.

5. The method according to claim 4, wherein said altered structure is a structure altered relative to that determined from analysis of a TOI image of said posterior segment obtained from a similarly situated subject known not to have the disease or known not to be at risk of developing the disease.

6. The method of claim 4 wherein determination of the maintenance of the altered structure between (a) and (b) is (d) a determination where said altered structure is substantially unchanged between (a) and (b); or (e) a determination where any further alteration or progression of said altered structure between (a) and (b) is not as severe or advanced as that between a set of reference first and second TOI images of said posterior segment obtained from a similarly situated subject known to have the disease and which subject has not been treated for said disease, wherein the reference first and second images were obtained or are to be obtained at the same interval as the first and second images of (a) and (b).

7. The method according to claim 4, wherein said second image is to be obtained at least 2 days and no more than 730 days subsequent to said first image.

8. The method according to claim 1, wherein said disease associated with an altered structure in the posterior segment of the eye is uveitis, glaucoma, macular edema, macular hole, macular pucker, diabetic macular edema, diabetic retinopathy, diabetic eye diseases, retinopathy, age-related macular degeneration (AMD), wet AMD, dry AMD, early AMD, intermediate AMD, central serous chorioretinopathy, scleritis, optic nerve degeneration, geographic atrophy, choroidal disease, ocular sarcoidosis, optic neuritis, choroidal neovascularization, retinitis pigmentosa, retinal tears, Stargardt disease, ocular cancer, retinitis, corneal ulcers, cataract, infection with a virus, infection with fungi, parasitic infection, bacterial infection, sarcoidosis, retinal vein occlusion, central retinal vein occlusion, branch retinal vein occlusion, retinal vascular disease, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, iridocyclitis diabetic retinopathy, retinopathy of prematurity (ROP), ischemic vasculopathies, inherited retinal dystrophies, retinal detachment, aberrant angiogenesis, retinal angiomatous proliferation (RAP), intraretinal microvascular abnormalities, pre-retinal neovascularization, choroidal angiogenesis, choroidal vasculopathy stroke, hypertension, diabetes, cardiovascular disease, prematurity, and papilloedema.

9. The method according to claim 1, wherein the image of said posterior segment of the eye is an image of the choroid, the choriocapillaris, Bruch's membrane, retinochoroidal tissue, the neuroretinal tissue, the nerve fiber layer, the retinal pigment epithelium (RPE), the photorepectors, the ganglion cell layer, the retinal vasculature, the subretinal space, the retina, the macula, the lamina cribrosa, the optic disc or the optic nerve.

10. The method according to claim , wherein said altered structure is an alteration in the tissue structure.

11. The method according to claim 10, wherein the alteration in tissue structure is an alteration in cell pattern, cell density, cell size, cell distribution or cell reflectivity.

12. The method according to claim 10, wherein said alteration in tissue structure is an alteration in tissue reflectivity that is an alteration in hyporeflective regions, hyperreflective regions, hyporeflective regions within a hyperreflective region, or any combination thereof.

13. The method according to claim 1, wherein said image is an image of the retinal pigment epithelium (RPE).

14. The method according to claim 1, wherein said image is an image of the choriocapillaris.

15. The method according to claim 1, wherein the image is an image of the nerve fiber layer, optic disc and/or retinal vasculature.

16. The method according to claim 13, wherein said disease is age-related macular degeneration (AMD) and said altered structure is an alteration in RPE cell density, RPE cell size, hyperreflective regions, hyporeflective regions, and/or hyporeflective regions within a hyperreflective region.

17. The method according to claim 13, wherein said disease is central serous chorioretinopathy and said altered structure is an alteration of the RPE and the choriocapillaris.

18. The method according to claim 15, wherein said disease is glaucoma and said altered structure is an alteration of the nerve fiber layer, or alteration of the optic disc morphology.

19. The method according to claim 15, wherein said disease is diabetic retinopathy and the altered structure is an alteration of the retinal vasculature.

20. The method according to claim 1, wherein said altered structure is indicative of geographic atrophy, drusen, reticular pseudo-drusen, neovasculature and/or retinal pigment epithelium degeneration.

21.-40. (canceled)
